(19) United States
(12) Patent Application Publication
(10) Pub. No.: US 2023/0052534 A1
(43) Pub. Date: Feb. 16, 2023

(54) CLASSIFIER OF EPILEPTIC NETWORK DYNAMICS

(71) Applicant: THE REGENTS OF THE UNIVERSITY OF MICHIGAN, Ann Arbor, MI (US)

(72) Inventors: William C. Stacey, Saline, MI (US); Dakota Crisp, Ann Arbor, MI (US)

(21) Appl. No.: 17/888,237

(22) Filed: Aug. 15, 2022

Related U.S. Application Data
(60) Provisional application No. 63/233,624, filed on Aug. 16, 2021.

(51) Int. Cl. A61N 1/36

(52) U.S. Cl. CPC A61N 1/36064; A61N 1/36025; A61N 1/36031; A61N 1/36135

(57) ABSTRACT

In some embodiments, an electrical probing stimulation pattern is delivered to the brain of a subject. A response to the electrical probing is analyzed, and used to determine a type of predicted seizure. The type of predicted seizure may be used to determine a treatment electrical stimulation pattern that may be administered to prevent onset of the predicted seizure. In some embodiments, a predicted seizure metric is calculated, which, in some implementations, acts as an indicator of “distance” (e.g., probability distance) to the predicted seizure. Furthermore, a subject model may be trained to assist with determining the type of predicted seizure, and determining the treatment electrical stimulation pattern.

### CROSS-REFERENCE TO RELATED APPLICATIONS

This application claims priority to U.S. Provisional Patent Application No. 63/233,624, filed Aug. 16, 2021, which is hereby incorporated by reference in its entirety.

### FIELD OF THE DISCLOSURE

The present disclosure relates generally to techniques for treating seizures and, more particularly, to techniques for analyzing electroencephalogram (EEG) signals to predict seizures, and to preventing the predicted seizures through electrical stimulation or other methods.

### BACKGROUND

The background description provided herein is for the purpose of generally presenting the context of the disclosure. Work of the presently named inventor, to the extent it is described in this background section, as well as aspects of the description that may not otherwise qualify as prior art at the time of filing, are neither expressly nor impliedly admitted as prior art against the present disclosure.

Clinicians and researchers are searching for new, more effective methods to treat seizures because existing techniques have certain drawbacks. For example, in the current state of the art, electrical stimulation is sometimes used to treat a seizure once the seizure has begun. This treatment sometimes effectively stops the seizure, and other times fails to stop the seizure; and it is not known why this treatment is effective for some seizures, but not others (or for some patient, but not others).

Another existing technique to treat seizures is through medication. However, to determine if a particular medication is effectively reducing the number of seizures that a patient has, it may take an inordinate amount of time to gather the required data (e.g., months).

The systems and methods disclosed herein improve upon these existing techniques and others.

### SUMMARY OF THE INVENTION

Techniques are described for analyzing EEG signals to predict seizures, and for preventing the predicted seizures through electrical stimulation or other methods. To assist with predicting a seizure (and type of seizure), a probing electrical stimulation pattern may be delivered to the subject (e.g., human patient). In addition, in some examples, a patient model is trained to assist with predicting the seizure and type of seizure, as well as to assist with determining a treatment electrical stimulation pattern.

In accordance with an example, a method comprises: sending, by a signal processing device, instructions to administer a probing electrical stimulation pattern to a subject through a plurality of electrodes; receiving, at the signal processing device, neuronal electrical activity signal data taken from the plurality of electrodes in response to the sending of the instructions to administer the probing electrical stimulation pattern; analyzing, in the signal processing device, the received neuronal electrical activity signal data and, from the received neuronal electrical activity signal, determining a type of predicted seizure from a plurality of seizure types; and determining, in the signal processing device, a treatment electrical stimulation pattern corresponding to the type of the predicted seizure for preventing the predicted seizure.

In some examples, the analyzing the received neuronal electrical activity signal data to determine the type of the predicted seizure comprises: identifying one or more seizure onset signal patterns in the received neuronal electrical activity signal data; generating a predicted seizure metric from the one or more seizure onset signal patterns, wherein the predicted seizure metric spans a plurality of value ranges each value range corresponding to a different one of the plurality of predicted seizure types; and determining the type of the predicted seizure based on the value range of the predicted seizure metric.

In some embodiments, the analyzing the received neuronal electrical activity signal data to determine the type of the predicted seizure comprises: identifying one or more seizure onset signal patterns in the received neuronal electrical activity signal data; generating a predicted seizure metric from the one or more seizure onset signal patterns, wherein the predicted seizure metric is a probability metric indicating a probability of the presence of each of the plurality of predicted seizure types; and determining the type of the predicted seizure based on the predicted seizure metric.

In some implementations, the analyzing the received neuronal electrical activity signal data to determine the type of the predicted seizure comprises: identifying one or more seizure onset signal patterns in the received neuronal electrical activity signal data, wherein the one or more seizure onset signal patterns are pre-bifurcation signal patterns.

In some examples, the analyzing the received neuronal electrical activity signal data to determine the type of the predicted seizure comprises: identifying one or more seizure onset signal patterns in the received neuronal electrical activity signal data, wherein the one or more seizure onset signal patterns include one or more signal bifurcation patterns.

In some embodiments, the analyzing the received neuronal electrical activity signal data to determine the type of the predicted seizure comprises: determining a plurality of seizure metrics, each seizure metric of the plurality of seizure metrics corresponding to a particular type of predicted seizure; and determining the type of predicted seizure according to the determined plurality of seizure metrics.

In some implementations, the analyzing the received neuronal electrical activity signal data to determine the type of the predicted seizure comprises: (i) determining a predicted seizure metric, and (ii) determining the type of the predicted seizure based on the predicted seizure metric; the method further comprises: in response to administering the treatment electrical stimulation pattern to the subject through the plurality of electrodes, receiving, in the signal processing device, further neuronal electrical activity signal data taken from a plurality of electrodes; determining, in the signal processing device, an increase, decrease, or no change in the predicted seizure metric based on the further neuronal electrical activity signal data and generating an updated predicted seizure metric; and in response to the determination of the increase, decrease, or no change in the predicted seizure metric, changing the treatment electrical stimulation to reduce the likelihood of the predicted seizure.

In some examples, the method further comprises: prior to the sending of the instructions to administer the probing stimulation pattern, receiving, via the signal processing device, training neuronal electrical activity signal data taken from the plurality of electrodes; and training, via the signal processing device, a model of the subject based on the received training neuronal electrical activity signal data; wherein the determination of the type of predicted seizure is further based on the trained subject model of the subject by: (i) inputting the neuronal electrical activity signal data into the trained patient model, and (ii) receiving the type of predicted seizure as an output of the trained patient model.

In some embodiments, the method further comprises administering the probing electrical stimulation pattern and/or the treatment electrical stimulation pattern to the subject through the plurality of electrodes.

In some implementations, the type of the predicted seizure is one of: a supercritical Hopf bifurcation (SupH) bifurcation; a Saddle-Node on an Invariant Circle (SNIC) bifurcation; a Saddle-Node (SN) bifurcation; or a Subcritical Hopf (SubH) bifurcation.

In some examples, the probing electrical stimulation pattern comprises at least one of: a brain location to stimulate; amplitude of the probing electrical stimulation pattern; frequency of the probing electrical stimulation pattern; duration of the probing electrical stimulation pattern; or start time of the probing electrical stimulation pattern.

In some embodiments, the treatment electrical stimulation pattern comprises at least one of: a brain location to stimulate; amplitude of the treatment electrical stimulation pattern; frequency of the treatment electrical stimulation pattern; duration of the treatment electrical stimulation pattern; or start time of the treatment electrical stimulation pattern.

In some implementations, the plurality of electrodes include at least one electrode configured to both: (i) administer at least part of the probing electrical stimulation pattern, and (ii) sense neuronal electrical activity.

In some examples, the plurality of electrodes includes: (i) an administration electrode configured to administer at least part of the probing electrical stimulation pattern, but not configured to sense neuronal electrical activity; and (ii) a sensing electrode configured to sense the neuronal electrical activity, but not configured to administer the probing electrical stimulation pattern.

In some embodiments, the plurality of electrodes are one or both of: (i) comprised in a therapeutic brain implant, or (ii) extracranial.

In some implementations, the sending of the instructions to administer the probing electrical stimulation pattern occurs subsequent to a first seizure.

In another example, a method comprises: sending, by a signal processing device, instructions to administer a probing electrical stimulation pattern to a subject through a plurality of electrodes; receiving, at the signal processing device, neuronal electrical activity signal data taken from the plurality of electrodes in response to the sent instructions to administer the probing electrical stimulation pattern; analyzing, in the signal processing device, the received neuronal electrical activity signal data and, from the received neuronal electrical activity signal, determining a type of predicted seizure from a plurality of seizure types; and determining, in the signal processing device, from the type of predicted seizure whether to administer a treatment electrical stimulation pattern to prevent the predicted seizure.

In some examples, the analyzing the received neuronal electrical activity signal data to determine the type of the predicted seizure comprises: identifying one or more seizure onset signal patterns in the received neuronal electrical activity signal data; generating a predicted seizure metric from the one or more seizure onset signal patterns, wherein the predicted seizure metric spans a plurality of value ranges each value range corresponding to a different one of the plurality of predicted seizure types; and determining the type of the predicted seizure based on the value range of the predicted seizure metric.

In some embodiments, the analyzing the received neuronal electrical activity signal data to determine the type of the predicted seizure comprises: identifying one or more seizure onset signal patterns in the received neuronal electrical activity signal data; generating a predicted seizure metric from the one or more seizure onset signal patterns, wherein the predicted seizure metric is a probability metric indicating a probability of the presence of each of the plurality of predicted seizure types; and determining the type of the predicted seizure based on the predicted seizure metric.

In some implementations, the determining whether to administer the treatment electrical stimulation pattern comprises determining to administer the treatment electrical stimulation pattern; and the method further comprises determining the treatment electrical stimulation pattern based on the determined type of predicted seizure, wherein the treatment electrical stimulation pattern comprises at least one of: a brain location to stimulate; amplitude of the treatment electrical stimulation pattern; frequency of the treatment electrical stimulation pattern; duration of the treatment electrical stimulation pattern; or start time of the treatment electrical stimulation pattern.

In some examples, the determining whether to administer the treatment electrical stimulation pattern comprises determining not to administer the treatment electrical stimulation pattern to prevent the predicted seizure, and wherein the method further comprises: in response to the determination that the treatment electrical stimulation pattern will not be administered, sending a warning message to a smartphone of a subject indicating: (i) the type of the predicted seizure, and (ii) that no electrical stimulation will be administered to prevent an onset of the predicted seizure.

In yet another example, a method comprises: receiving, via one or more processors, training neuronal electrical activity signal data taken from a plurality of electrodes; receiving, via the one or more processors, medication data of a subject, wherein the medication data includes a type of a medication, a dosage amount of the medication, and a time that the medication was administered to the subject; and training, via the one or more processors, a subject model of the subject based on: (i) the received training neuronal electrical activity signal data, and (ii) the medication data of the subject; wherein the subject model is operable to determine a type of seizure based on subsequent neuronal electrical activity signal data.

In some examples, the method further comprises: receiving, via the one or more processors, brain state data of the subject.

In some embodiments, the brain state data includes at least one of: sleep stage data of the subject or circadian rhythm data of the subject.

In some implementations, the type of the predicted seizure is one of: a supercritical Hopf bifurcation (SupH) bifurcation; a Saddle-Node on an Invariant Circle (SNIC) bifurcation; a Saddle-Node (SN) bifurcation; or a Subcritical Hopf (SubH) bifurcation.

In some examples, the one or more processors are comprised in a signal processing device, and the method further includes: sending, by the signal processing device, instructions to administer a probing electrical stimulation pattern to the subject through the plurality of electrodes; in response to the sending of the instructions to administer the probing electrical stimulation pattern, receiving, at the signal processing device, the subsequent neuronal electrical activity signal data; determining, in the signal processing device, a type of the predicted seizure by inputting the subsequent neuronal electrical activity signal data into the trained model; determining, in the signal processing device, a treatment electrical stimulation pattern to prevent the predicted seizure based on the type of the predicted seizure; administering, through the plurality of electrodes, the treatment electrical stimulation pattern; and updating, in the one or more processors, the subject model based on neuronal electrical activity signal data taken from the plurality of electrodes in response to the administered treatment electrical stimulation pattern.

### BRIEF DESCRIPTION OF THE DRAWINGS

The figures described below depict various aspects of the system and methods disclosed herein. It should be understood that each figure depicts an embodiment of a particular aspect of the disclosed system and methods, and that each of the figures is intended to accord with a possible embodiment thereof. Further, wherever possible, the following description refers to the reference numerals included in the following figures, in which features depicted in multiple figures are designated with consistent reference numerals.

FIG. 1 illustrates an example system for predicting a seizure, and/or treating a seizure.

FIG. 2A illustrates an example device comprising extracranial electrodes.

FIG. 2B illustrates an example device comprising electrodes implanted into a patient.

FIG. 3 illustrates example onset and offset bifurcations.

FIG. 4 illustrates a flow diagram of a high level of an example method including delivering a probing electrical stimulation pattern.

FIG. 5 illustrates a flow diagram of an example embodiment including a determination of whether to provide electrical stimulation treatment.

FIG. 6 illustrates a flow diagram of an example embodiment including closed loop feedback.

FIG. 7 illustrates an example method including training a patient model.

### DETAILED DESCRIPTION

Provided are techniques for analyzing EEG signals to predict seizures, and techniques for preventing the predicted seizures through electrical stimulation or other methods. The techniques, which may be implemented in software and/or hardware and which may be fully or partially automated, offer a number of advantages including: (i) stopping a seizure from occurring before it begins; (ii) stopping a seizure once it has begun more effectively than prior techniques; and (iii) gathering the data required to build an effective treatment plan for a patient in less time than prior techniques.

### Example System Overview

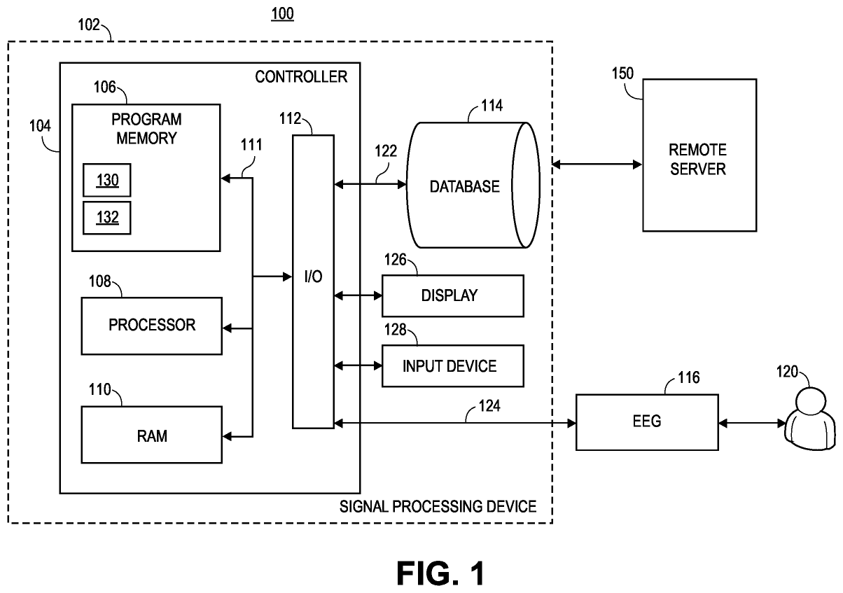

FIG. 1 illustrates an example block diagram 100 illustrating the various components used in implementing an example embodiment of the present techniques. A signal processing device 102 (or “signal processor” or “diagnostic device”) is configured to collect EEG data taken and/or deliver electrical stimulation to or from a patient 120 via an EEG device 116 (electrodes not shown in FIG. 1) in accordance with executing the functions of the disclosed embodiments. The signal processing device 102 may have a controller 104 operatively connected to a database 114 via a link 122 connected to an input/output (I/O) circuit 112. It should be noted that, while not shown, additional databases may be linked to the controller 104 in a known manner. The controller 104 includes a program memory 106, one or more processors 108 (may be called microcontrollers or a microprocessors), a random-access memory (RAM) 110, and the input/output (I/O) circuit 112, all of which are interconnected via an address/data bus 111. It should be appreciated that although only one processor 108 is shown, the controller 104 may include multiple microprocessors 108. Similarly, the memory of the controller 104 may include multiple RAMs 110 and multiple program memories 106. Although the I/O circuit 112 is shown as a single block, it should be appreciated that the I/O circuit 112 may include a number of different types of I/O circuits. The RAM(s) 110 and the program memories 106 may be implemented as semiconductor memories, magnetically readable memories, and/or optically readable memories, for example. A link 124, which may include one or more wired and/or wireless (Bluetooth, WLAN, etc.) connections, may operatively connect the controller 104 to the EEG device 116 through the I/O circuit 112. In other examples, the EEG device 116 may be part of the signal processing device 102.

The program memory 106 and/or the RAM 110 may store various applications (i.e., machine readable instructions) for execution by the processor 108. For example, an operating system 130 may generally control the operation of the signal processing device 102 and provide a user interface to the signal processing device 102 to implement data processing operations. The program memory 106 and/or the RAM 110 may also store a variety of subroutines 132 for accessing specific functions of the signal processing device 102. By way of example, and without limitation, the subroutines 132 may include, among other things: a subroutine for gathering EEG data from the device 116, a subroutine for building and/or updating a patient model (e.g., of the patient 120), a subroutine for calculating an electrical stimulation pattern to deliver to the patient 120, and/or a subroutine for delivering electrical stimulation through the device 116.

The subroutines 132 may also include other subroutines, for example, implementing software keyboard functionality, interfacing with other hardware in the signal processing device 102, etc. The program memory 106 and/or the RAM 110 may further store data related to the configuration and/or operation of the signal processing device 102, and/or related to the operation of the one or more subroutines 132. For example, the data may be data gathered by the device 116, data determined and/or calculated by the processor 108, etc. In addition to the controller 104, the signal processing device 102 may include other hardware resources. The signal processing device 102 may also include various types of input/output hardware such as a visual display 126 and input device(s) 128 (e.g., keypad, keyboard, etc.). In an embodiment, the display 126 is touch-sensitive, and may cooperate with a software keyboard routine as one of the software routines 132 to accept user input.

It may be advantageous for the signal processing device 102 to communicate with a medical treatment device, medical data records storage device, or network (not shown) through any of a number of known networking devices and techniques (e.g., through a computer network such as a hospital or clinic intranet, the Internet, etc.). For example, the signal processing device may be connected to a medical records database, hospital management processing system, healthcare professional terminals (e.g., doctor stations, nurse stations), patient monitoring systems, automated drug delivery systems such as smart pumps, smart infusion systems, automated drug delivery systems, etc. Accordingly, the disclosed embodiments may be used as part of an automated closed loop system or as part of a decision assist system.

Additionally or alternatively, in some embodiments, the signal processing device 102 communicates with remote server 150, which may comprise processor(s), memory, etc. In some embodiments, the remote server 150 comprises a database used to store patient data (e.g., of the patient 120) and/or processing device(s) used to train a model of the patient 120. The signal processing device 102 and remote server 150 may communicate via any suitable technique. For example, the processing device 102 and remote server 150 may communicate via the internet, a hospital or clinic intranet, a cellular network, Bluetooth, and/or any other suitable method.

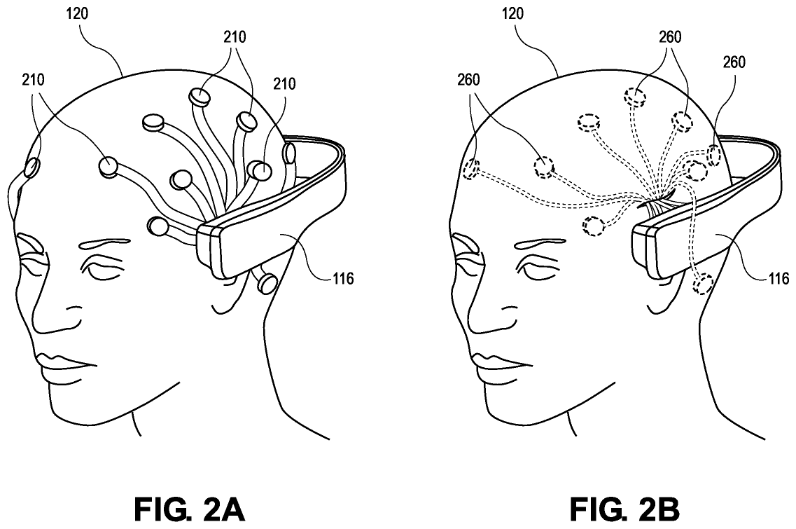

Although depicted as separate entities or components in FIG. 1, it is understood that any or all of the signal processing functionality and/or components of the signal processing device 102 may be combined with an EEG device 116, such as in the examples of FIGS. 2A and 2B. In this manner, a system 100 may both gather EEG about the patient 120, e.g., through extracranial electrodes 210 and/or intracranial electrodes 260, and process the gathered data to identify and analyze one or more features thereof. The system 100 may further deliver electrical stimulation to the patient 120 (e.g., probing electrical stimulation and/or treatment electrical stimulation). Also, although depicted as a single component in FIG. 1, the EEG 116 may include multiple of the same type or different types of brain activity measuring devices. In some embodiments, the present techniques may be integrated into existing EEG devices, including EEG software display devices.

FIG. 2A illustrates an example EEG device 116 comprising extracranial electrodes 210, which may perform any of the functions discusses herein. For example, the extracranial electrodes 210 may sense electrical signals from the patient 120. Additionally or alternatively, the extracranial electrodes 210 may deliver an electrical stimulation pattern to the patient 120. Although in some embodiments all of the extracranial electrodes 210 may perform the same function(s) (e.g., both measuring the patient's 120 brain activity, and delivering electrical treatment), in other embodiments, the extracranial electrodes 210 comprise groups of electrodes dedicated for particular functions. For example, one group of electrodes senses signals, while another delivers electrical stimulation, while still another both senses signals and delivers electrical stimulation.

FIG. 2B illustrates an example EEG device 116 comprising electrodes 260 implanted into a patient 120. For instance, the implanted electrodes 260 may sense electrical signals from the patient 120. Additionally or alternatively, the implanted electrodes 260 may deliver an electrical stimulation pattern to the patient 120. Although in some embodiments all of the implanted electrodes 260 may perform the same function(s) (e.g., both measuring the patient's 120 brain activity, and delivering electrical treatment), in other embodiments, the implanted electrodes 260 comprise groups of electrodes dedicated for particular functions. For example, one group of electrodes senses signals, while another delivers electrical stimulation, while still another both senses signals and delivers electrical stimulation.

Furthermore, some embodiments include an EEG device 116 that is a combination of the example of FIGS. 2A and 2B. For example, some embodiments include an EEG device that comprises both extracranial electrodes 210, and implanted electrodes 260.

### Example Methods

By way of broad overview, as a seizure occurs, develops, and ends, the brain moves from a normal state into a seizure and back again; and this brain activity may be tracked and analyzed in accordance with the principles described herein. Furthermore, at the onset and offset of the seizure, there are sudden qualitative changes in brain activity known as “bifurcations.” However, not all bifurcations produce the same brain activity changes, thereby allowing the bifurcations to be classified into different types (e.g., different types of onset bifurcations, offset bifurcations; or simply different “types of seizures”).

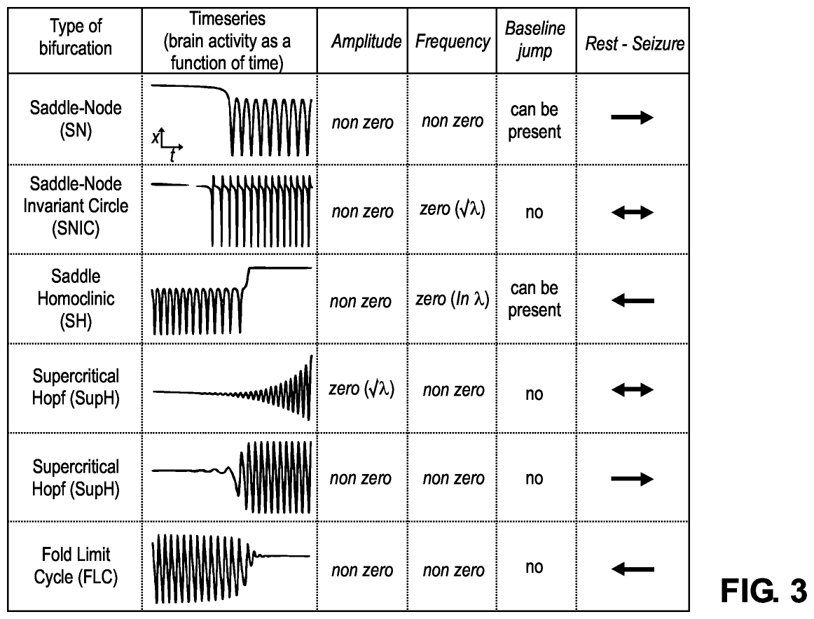

In this regard, FIG. 3 illustrates example onset and offset bifurcations. In particular, FIG. 3 illustrates four onset bifurcation types: saddle-node (SN) bifurcation; saddle-node on an invariant circle (SNIC) bifurcation; supercritical hopf bifurcation (SupH) bifurcation; and subcritical hopf (SubH) bifurcation. FIG. 3 further illustrates two offset bifurcation types: saddle homoclinic (SH) bifurcation; and fold limit cycle (FLC) bifurcation.

In this respect, for each type of bifurcation, the example of FIG. 3 illustrates: a timeseries (electrical brain activity as a function of time); amplitude of the brain activity; frequency of the brain activity; baseline jump; and rest-seizure. As illustrated, seizures may be classified into different types based on the electrical activity measured from the brain of patient 120 (e.g., based on any of the information illustrated in the example of FIG. 3). For example, a seizure with a nonzero amplitude, a nonzero frequency, and a baseline jump would be classified as SN. In another example, a seizure with an amplitude of zero(\sqrt{λ}) would be classified as SupH.

Further regarding the four onset bifurcation types illustrated in the example of FIG. 3, two of the onset bifurcation types (SupH, and SNIC) progress gradually from the resting state into seizure by scaling laws for the amplitude or frequency. More specifically, in SupH, the amplitude of the oscillations starts at zero, and increases proportionally to the square root of the distance from the bifurcation point. And, the SNIC bifurcation has oscillations that increase in frequency as the square-root of the same distance.

On the other hand, SN and SubH have abrupt amplitude and frequency changes that do not follow specific scaling laws. SN sometimes includes a jump in the signal baseline (i.e., direct current (DC) shift), but, in the absence of a detectable DC shift, SN and SubH may be difficult to distinguish. SN without DC shift and SubH are thus sometimes grouped together.

However, it should be understood that once a patient 120 exhibits the brain activity shown in the onset bifurcations illustrated in FIG. 3, the seizure has already begun. Although treatment (e.g., electrical stimulation) may be administered at this stage, it may be more useful to administer treatment even before the seizure has begun, and thereby prevent the seizure from occurring in the first place. Thus, some embodiments predict the likelihood of a seizure (or a specific type of seizure) occurring. In this regard, a predicted seizure metric may be used in determining a patient's seizure risk. In some embodiments, the predicted seizure metric is not a probability itself; rather, the predicted seizure metric is a general indicator. In some respects, this is analogous to high blood pressure being an indicator for a heart attack—the high blood pressure itself is not a probability, but rather is a general indicator that a patient may be at an increased risk for heart attack. Thus, in one aspect, the predicted seizure metric acts as an indicator of “distance” (e.g., probability distance) to a seizure. Yet, in other embodiments, the predicted seizure metric is a numerical indicator of probability that the predicted seizure will occur.

Further regarding the predicted seizure metric, in some embodiments, the predicted seizure metric may be at least partially determined by leveraging electrical probing. That is, a probing electrical stimulation pattern may be delivered to a patient 120, and the response may be measured. The response may then be used to (wholly or spatially) determine the predicted seizure metric.

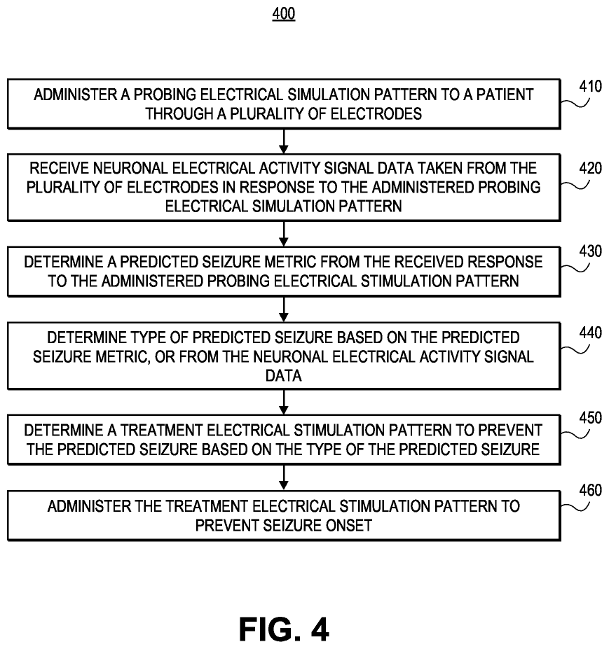

In this regard, FIG. 4 provides a flow diagram of a high level of an example method 400. With reference thereto, at block 410, a probing electrical stimulation pattern is delivered to the patient 120 through the plurality of electrodes 210, 260. In some embodiments, this involves the signal processing device 102 controlling the EEG device 116 to deliver the probing electrical stimulation pattern (e.g., the signal processing device 120 determining and sending instructions to deliver the probing electrical stimulation pattern). In some embodiments, the probing electrical stimulation pattern delivered at block 410 is delivered in the time period between seizures, known as the “interictal” time period. In this regard, in some embodiments, the seizures themselves are identified (e.g., based on the characteristics illustrated in the example of FIG. 3) without probing; whereas, the electrical probing is used to predict possible future seizure(s).

The probing electrical stimulation pattern may comprise any or all of: a brain location to stimulate; amplitude of the electrical stimulation pattern; frequency of the electrical stimulation pattern; duration of the electrical stimulation pattern; start time of the electrical stimulation pattern; and/or end time of the electrical stimulation pattern. The probing electrical stimulation pattern may be determined by any suitable technique. For instance, the probing electrical stimulation pattern may be a generic electrical stimulation pattern that is used for all patients. Alternatively, the probing electrical stimulation pattern may be based on characteristics of the patient (e.g., gender, age, height, weight, medical history including history of seizures, etc.). Additionally or alternatively, the probing electrical stimulation pattern may be based on a patient model, and the training of the patient model will be described further below in later sections.

At block 420, a response to the electrical probing stimulation pattern is received. In this regard, the electrical probing may be used to determine the type of the predicted seizure. This may be done directly (e.g., by analyzing the response to the electrical probe to determine the type of predicted seizure), or through the predicted seizure metric (e.g., by determining the predicted seizure metric, e.g., at block 430, from the response to the electrical probe; and then determining the type of predicted seizure from the predicted seizure metric, such as at block 440). In some embodiments, the type of seizure may be: SN, SNIC, SupH, or SubH.

In one example, the predicted seizure metric is a number (which may or may not be a numeric indicator of probability of the seizure occurring) that is first determined from the response to the electrical probe; then, depending on what range the predicted seizure metric falls into, the type of seizure is determined. However, in another example, the predicted seizure metric is not a single number, and rather is a collection of data that is gathered from the analysis of neuronal electrical activity signal data of the patient 120.

Advantageously, one contribution of the present disclosure is the discovery that different types of seizures respond differently to electrical probing. The systems and methods described herein leverage this discovery by analyzing the response to the electrical probing to determine the type of predicted seizure. In this regard, in some embodiments, the predicted seizure metric comprises individual metrics for each type of seizure. For example, the predicted seizure metric may comprise four different numbers, one for each of SN, SNIC, SupH, and SubH. Furthermore, it may be noted that it is possible for the patient 120 to have different types of seizures.

To further elaborate on the predicted seizure metric, in some embodiments, the predicted seizure metric is a number or set of numbers that is determined based on the received response to the electrical probe. The predicted seizure metric may further be determined based on other factors, such as characteristics of the patient (e.g., gender, age, height, weight, medical history including history of seizures, etc.). Additionally or alternatively, the predicted seizure metric may be calculated based on the trained patient model, as will be described in more detail in the following sections.

Still further regarding the predicted seizure metric, some embodiments analyze “seizure onset signal” patterns in the patient's 120 brain activity to determine (wholly or partially) the predicted seizure metric. In this regard, the brain activity illustrated in the example of FIG. 3 shows brain activity when the seizure has already substantially begun. Seizure onset signal, on the other hand, are patterns in the patient's 120 brain activity that occur before the seizure (or bifurcation) has begun (e.g., seconds, minutes, hours, or days before the seizure has begun).

Data of seizure onset signal patterns, in some embodiments, is stored in the program memory 106 and/or database 114. More specifically, in some embodiments, neuronal electrical activity signal data taken from the patient 120 is compared with the data of the seizure onset signal patterns stored in the memory 106 and/or database 114 as part of the calculation of the predicted seizure metric. Additionally or alternatively, over time, patient-specific seizure onset signal patterns may be determined and added to the program memory 106 and/or database 114. Furthermore, the patient-specific seizure onset signal patterns may be used in training the model of the patient. It should be understood that, as part of the calculation of the predicted seizure metric or training of the model of the patient, the seizure onset signal patterns may be analyzed based on amplitude, frequency, brain location of occurrence, or any other suitable criteria. Similarly, these characteristics of the seizure onset signal patterns may be used to determine the type of predicated seizure without use of the predicted seizure metric (e.g., by analyzing the waveforms to determine if seizure onset signal patterns are occurring).

At block 450, a treatment electrical stimulation pattern is determined (e.g., by the signal processing device 102) to prevent the predicted seizure. The treatment electrical stimulation pattern may be determined based on the predicted type of seizure, and may be done in any suitable manner. For example, the signal processing device may have preset treatment electrical stimulation patterns (e.g., stored in the memory 106) corresponding to the types of seizures. The preset patterns may be used directly, or may be modified based on patient characteristics (e.g., gender, age, height, weight, medical history including history of seizures, etc.), and/or the trained model of the patient. In some embodiments, there is no preset pattern, and the treatment electrical stimulation pattern is determined from the received response to the electrical probing.

The treatment electrical stimulation pattern may comprise any or all of: a brain location to stimulate; amplitude of the electrical stimulation pattern; frequency of the electrical stimulation pattern; duration of the electrical stimulation pattern; start time of the electrical stimulation pattern; and/or end time of the electrical stimulation pattern.

At block 460, the treatment electrical stimulation pattern is delivered to the patient 120 (e.g., through electrodes 210, 260). In some embodiments, the same electrodes 210, 260 are used to both sense and to deliver treatment, while other embodiments use separate, dedicated groups of electrodes to sense and deliver treatment.

Moreover, to even better determine the “distance” (e.g., probability distance) to the predicted seizure, additional electrical probes may be delivered at any time. For example, following any of blocks 430, 440, 450 and/or 460, an additional electrical probe may be delivered to the patient 120. The predicted seizure metric may then be updated (or recalculated) based on the response to the additional probe, thereby enabling a determination of if the patient is moving “closer to” or “farther away from” the predicted seizure.

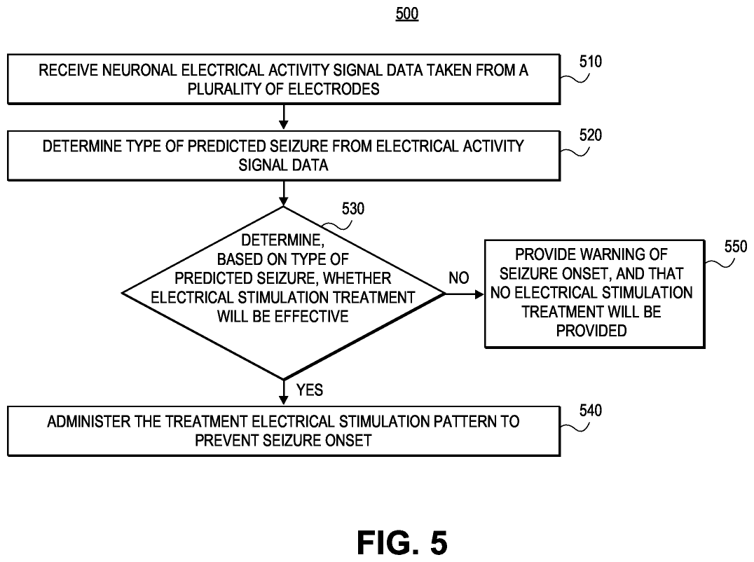

FIG. 5 shows a flowchart of an example embodiment including a determination of whether to provide electrical stimulation treatment. In this regard, it may be noted that, in prior art systems involving electrical stimulation treatment to stop a seizure, it was not known why the electrical stimulation treatment was sometimes effective and sometimes not effective in stopping the seizure. It is one contribution of the present application to explain that whether or not electrical stimulation treatment may be effective is because electrical stimulation treatment is effective for some types of seizures, but not others. Thus, some embodiments administer electrical stimulation treatment only when the type of predicted seizure can be prevented or mitigated by electrical stimulation. Accordingly, some embodiments provide a technical advantage over prior systems in that ineffective electrical treatment is avoided (e.g., if it is determined that electrical stimulation treatment will be ineffective, such treatment is not administered).

In any event, at block 510 of the example method 500, the signal processing device 102 receives neuronal electrical activity signal data taken from a plurality of electrodes 210, 260. In some embodiments, the neuronal electrical activity data is received in response to an electrical probe, such as a probe administered at block 410 from the example of FIG. 4. In other embodiments, no electrical probe is administered.

At block 520, the signal processing device 102 determines a type of predicted seizure from the electrical activity signal data. In some embodiments, the determination of the type of predicted seizure includes: (i) determining the predicted seizure metric from the received neuronal electrical activity signal data, and (ii) determining the type of predicted seizure based on the predicted seizure metric. In some implementations, the type of seizure may be: SN, SNIC, SupH, or SubH.

At block 530, the signal processing device 102 determines whether to administer a treatment electrical stimulation pattern to prevent the predicted seizure based on the type of the predicted seizure. For example, in some embodiments, if the predicted type of seizure is SN or SuPH, the determination is made to deliver electrical stimulation treatment; whereas, if the predicted type of seizure is SuBH, the determination is made to not deliver electrical stimulation treatment.

If the determination is made to deliver the electrical stimulation treatment, the electrical stimulation treatment pattern is delivered at block 540. As discussed above, the electrical stimulation treatment pattern may include any or all of: a brain location to stimulate; amplitude of the treatment electrical stimulation pattern; frequency of the treatment electrical stimulation pattern; duration of the treatment electrical stimulation pattern; or start time of the treatment electrical stimulation pattern.

If the determination is made not to deliver electrical stimulation treatment, a different course of action is taken at block 550. For example, a warning may be provided (e.g., to a smartphone of the patient 120, or to a medical device of a clinician) that a seizure has been predicted to occur, and that no electrical stimulation treatment will be provided. However, it should be understood that any other course of action (besides delivering electrical stimulation treatment) may be provided. For instance, an advisory may be sent for the patient to take medication.

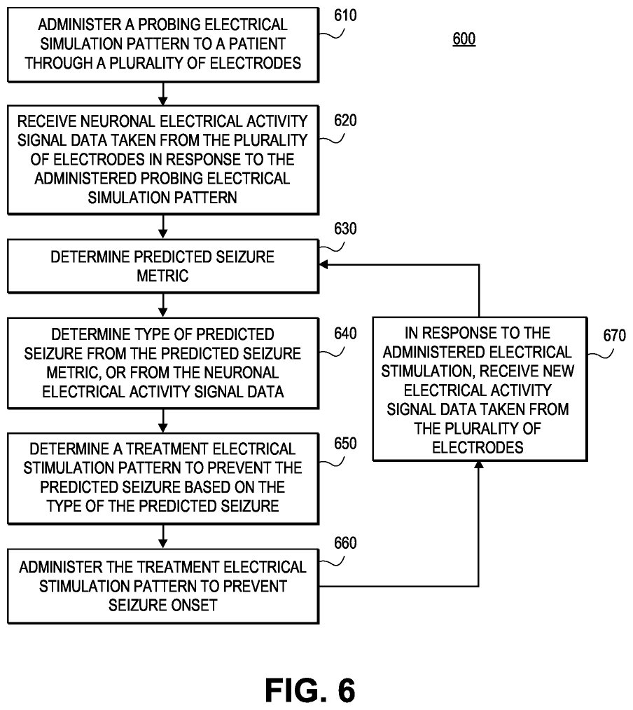

FIG. 6 illustrates an example method 600 including closed loop feedback. With reference thereto, at block 610, a probing electrical stimulation pattern is delivered to the patient 120 through the plurality of electrodes 210, 260. In some embodiments, this involves the signal processing device 102 controlling the EEG device 116 to deliver the probing electrical stimulation pattern (e.g., the signal processing device 120 determining and sending instructions to deliver the probing electrical stimulation pattern). In some embodiments, block 610 is similar to block 410 of the example of FIG. 4.

At block 620, the signal processing device 102 receives neuronal electrical activity signal data taken from the plurality of electrodes 210, 260 in response to the administered probing electrical stimulation pattern. In some embodiments, block 620 is similar to block 420 of the example of FIG. 4.

At block 630, the signal processing device 102 determines the predicted seizure metric. In some embodiments, block 630 is similar to block 430 of the example of FIG. 4. At block 640, the signal processing device 102 determines the type of predicted seizure from the predicted seizure metric, or from the neuronal electrical activity signal data. In some embodiments, block 640 is similar to block 440 of the example of FIG. 4.

At block 650, the signal processing device 102 determines a treatment electrical stimulation pattern to prevent the predicted seizure based on the type of the predicted seizure. In some embodiments, block 650 is similar to block 450 of the example of FIG. 4.

At block 660, the signal processing device 102 administers (through the electrodes 210, 260) the treatment electrical stimulation pattern to prevent seizure onset. In some embodiments, block 660 is similar to block 460 of the example of FIG. 4.

At block 670, the signal processing device revives new electrical activity signal data taken from the plurality of electrodes 210, 260 in response to the administered treatment electrical stimulation.

Subsequently, the method 500 returns (e.g., iterates) back to block 630. Here, the predicted seizure metric is recalculated and/or updated based on the response received at block 660. In this way, the system provides closed loop feedback. In addition, at any point throughout the method, additional electrical probes may be delivered. In this respect, at block 630, the predicted seizure metric may be recalculated or updated based on both: (i) the response received at block 660, and (ii) a received response to an additional probing electrical stimulation pattern. In this regard, in some embodiments, each response to each electrical probe acts as interictal biomarker.

Furthermore, this closed loop system allows for a determination of if the patient 120 is moving “closer to” or “farther away from” a predicted seizure. In other words, in some embodiments, when the predicted seizure metric is recalculated or updated at block 630, the signal processing device 120 may determine whether the predicted seizure is becoming more or less likely to occur.

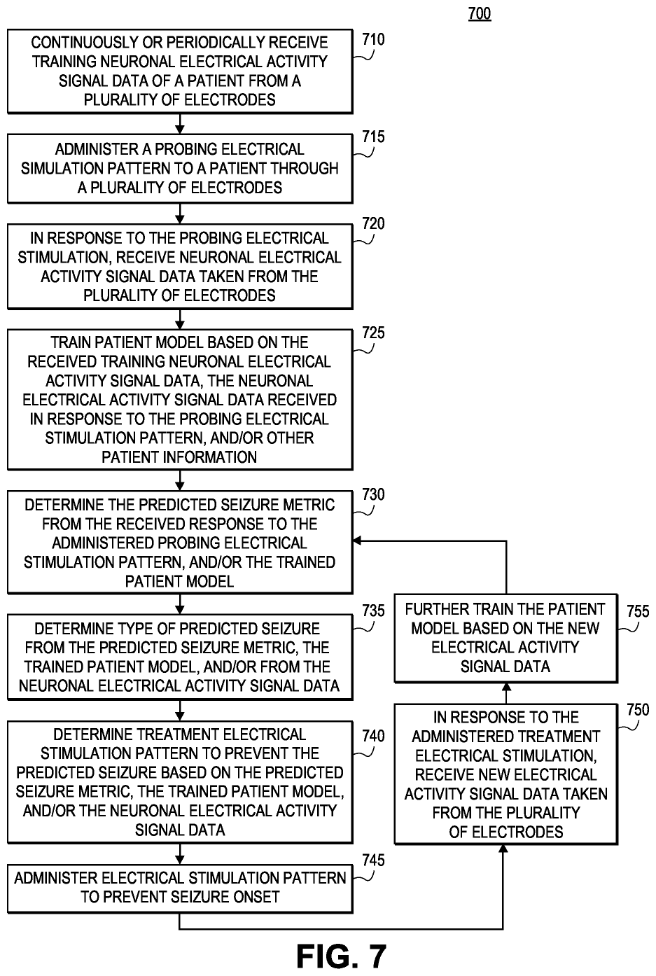

FIG. 7 illustrates an example method 700 including training a patient model. At block 710, the signal processing device 102 continuously or periodically receives training neuronal electrical activity signal data taken from the plurality of electrodes 210, 260. In some embodiments, the training neuronal electrical activity signal data is simply data that is taken while monitoring the patient 120. The data may be continuously gathered for a period of time, or periodically gathered at specific time intervals.

Furthermore, in some embodiments, the training neuronal electrical activity signal data is not taken from the plurality of electrodes, and rather, is simply uploaded from a database (e.g., a database of remote server 150). In other words, in some embodiments, the training neuronal electrical activity signal data is historical data (e.g., previous data, gathered by a different group of electrodes before the method 700 begins) simply uploaded from a database. For instance, a patient may have previous neuronal electrical activity signal data from previous hospital visits that is used as training data.

At block 715, a probing electrical stimulation pattern is delivered to the patient 120 through the plurality of electrodes 210, 260. In some embodiments, this involves the signal processing device 102 controlling the EEG device 116 to deliver the probing electrical stimulation pattern (e.g., the signal processing device 120 determining and sending instructions to deliver the probing electrical stimulation pattern). In some embodiments, block 715 is similar to block 410 of the example of FIG. 4.

At block 720, the signal processing device 102 receives neuronal electrical activity signal data taken from the plurality of electrodes 210, 260 in response to the administered probing electrical stimulation pattern. In some embodiments, block 720 is similar to block 420 of the example of FIG. 4.

At block 725, the signal processing device 102 (or any other processing device) trains the patient model based on the received training neuronal electrical activity signal data, the neuronal electrical activity signal data received in response to the probing electrical stimulation pattern, and/or other patient information. The patient model may be trained or built by any suitable technique. For instance, the patient model may simply be trained using the received training neuronal electrical activity signal data without using any artificial intelligence (AI) technique (e.g., by simply building the patient model). Additionally or alternatively, the patient model may be trained using an Al technique such as deep learning, neural networks, etc.

In one example, the patient model is trained by gathering data of when the patient 120 has seizures (e.g., by identifying patterns of specific types of seizures, such as those in the example of FIG. 3), and then identifying pre-bifurcation patterns in the patient's 120 brain activity. In this regard, it should be understood that, in some embodiments, the trained patient model may later be used such that it takes brain activity signals form the EEG 116 as inputs, and then outputs the predicted seizure metric.

Furthermore, it should be noted that the model may be trained by any suitable device. For example, the patient model may be trained by processor(s) via cloud computing. For instance, the data taken from the EEG 116 may be sent from the signal processing device 102 to the remote server 150 for the remote server to train the patient model.

As mentioned above, the patient model may be trained based on other patient information. The other patient information may be any information of the patient 120. In particular, medication data (e.g., including a type of a medication, a dosage amount of the medication, a time that the medication was administered to the patient 120, etc.); brain state data at particular times of the day (e.g., sleep stage data, circadian rhythm data); or aggregated sleep data (e.g., including an amount of time that the patient 120 spent sleeping); and/or alcohol intake data of the patient 120.

The other patient information may be received by the signal processing device 102 by any suitable method. For instance, signal processing device 102 may receive the other patient information from the remote server 150 (e.g., as independent information, or as part of other medical records). Additionally or alternatively, the patient may enter the other patient information into a smartphone or other computing device, and send it to the signal processing device 102. Additionally or alternatively, some or all of the other patient information may be automatically generated. For instance, the signal processing device 102 may, through analysis of information from the electrodes 210, 260, determine that the patient 120 is sleeping, and thereby automatically generate sleep data of the patient 120. Additionally or alternatively, the sleep data may be generated by analysis of video images of the patient 120.

Here, it may be noted that prior systems for determining an effective seizure medication relied on simply taking a patient's medication data, and comparing it to an amount of seizures that the patient had while taking the medication. While this could be effective in determining a suitable seizure medication for the patient 120, this process took a great amount of time (e.g., months). Advantageously, by building a patient model in accordance with the techniques disclosed herein, the amount of time it takes to determine an effective seizure medication for the patient 120 is greatly improved. For instance, by building the patient model based on both the medication data of the patient 120 and the training neuronal electrical activity signal data, it is possible to greatly reduce the amount of time that it takes to determine if a particular medication is effective.

At block 730, the signal processing device 102 uses the trained patient model to determine the predicted seizure metric. For example, the trained patient model may take the received response to the administered probing electrical stimulation pattern as an input, and then output the predicted seizure metric.

At block 735, the signal processing device 102 determines the type of predicted seizure from the predicted seizure metric. In some embodiments, block 735 is similar to blocks 440 and 640 from the preceding examples. However, the signal processing device 102 may determine the type of predicted seizure without calculating the predicted seizure metric. For example, the signal processing device 102 may simply analyze the response to the probing electrical stimulation to (with or without the trained patient model) determine the type of predicted seizure.

At block 740, the signal processing device 102 determines a treatment electrical stimulation pattern to prevent the predicted seizure based on the type of the predicted seizure and/or the trained patient model. In some embodiments, block 740 is similar to blocks 450 and 650 of the preceding examples. However, at block 740, because there is a trained patient model, the treatment electrical stimulation pattern may be determined wholly or partially based on the trained patient model (rather than, e.g., just the type of predicted seizure). For example, any aspect of the treatment electrical stimulation pattern may be determined based on the patient model (e.g., a brain location to stimulate; amplitude of the treatment electrical stimulation pattern; frequency of the treatment electrical stimulation pattern; duration of the treatment electrical stimulation pattern; or start time of the treatment electrical stimulation pattern, etc.).

At block 745, the signal processing device 102 administers (through the electrodes 210, 260) the treatment electrical stimulation pattern to prevent seizure onset. In some embodiments, block 745 is similar to blocks 460 and 660 of the preceding examples.

At block 750, the signal processing device 102 receives new electrical activity signal data taken from the plurality of electrodes 210, 260 in response to the administered treatment electrical stimulation.

At block 755, the patient model is further trained based on the response received at block 750. In this regard, the patient model is updated in real time. In addition, the patient model may be further trained based on any additional other patient information received. For instance, the patient may have switched medications, changed their sleeping patterns, and/or changed their alcohol intake; and this it may be beneficial to further train the patient model based on these updates.

The example method 700 the returns (e.g., iterates) back to block 730 where the predicted seizure metric is recalculated and/or updated (based on, e.g., the patient model that was further trained at block 755). As this shows, the system provides closed loop feedback. In addition, at any point throughout the method, additional electrical probes may be delivered. In this regard, at block 730, the predicted seizure metric may be recalculated and/or updated based on both: (i) the response received at block 750, and (ii) a received response to an additional probing electrical stimulation pattern.

Moreover, the example method 700 allows for a determination of if the patient 120 is moving “closer to” or “farther away from” a predicted seizure. Put another way, in some embodiments, when the predicted seizure metric is recalculated or updated at block 730, the signal processing device 120 may determine whether the predicted seizure is becoming more or less likely to occur.

Further regarding the example flowcharts provided above, it should be noted that all blocks are not necessarily required to be performed. Moreover, additional blocks may be performed although they are not specifically illustrated in the example flowcharts. In addition, the example flowcharts are not mutually exclusive. For example, block(s) from one example flowchart may be performed in another of the example flowcharts.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connects the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of the example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but also deployed across a number of machines. In some example embodiments, the processor or processors may be located in a single location (e.g., within a home environment, an office environment or as a server farm), while in other embodiments the processors may be distributed across a number of locations.

The performance of certain of the operations may be distributed among the one or more processors, not only residing within a single machine, but also deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

While the present invention has been described with reference to specific examples, which are intended to be illustrative only and not to be limiting of the invention, it will be apparent to those of ordinary skill in the art that changes, additions and/or deletions may be made to the disclosed embodiments without departing from the spirit and scope of the invention.

The foregoing description is given for clearness of understanding; and no unnecessary limitations should be understood therefrom, as modifications within the scope of the invention may be apparent to those having ordinary skill in the art.

What is claimed:

1. A method comprising:
sending, by a signal processing device, instructions to administer a probing electrical stimulation pattern to a subject through a plurality of electrodes;
receiving, at the signal processing device, neuronal electrical activity signal data taken from the plurality of electrodes in response to the sending of the instructions to administer the probing electrical stimulation pattern;
analyzing, in the signal processing device, the received neuronal electrical activity signal data and, from the received neuronal electrical activity signal, determining a type of predicted seizure from a plurality of seizure types; and
determining, in the signal processing device, a treatment electrical stimulation pattern corresponding to the type of the predicted seizure for preventing the predicted seizure.

2. The method of claim 1, wherein the analyzing the received neuronal electrical activity signal data to determine the type of the predicted seizure comprises:
identifying one or more seizure onset signal patterns in the received neuronal electrical activity signal data;
generating a predicted seizure metric from the one or more seizure onset signal patterns, wherein the predicted seizure metric spans a plurality of value ranges each value range corresponding to a different one of the plurality of predicted seizure types; and
determining the type of the predicted seizure based on the value range of the predicted seizure metric.

3. The method of claim 1, wherein:
the analyzing the received neuronal electrical activity signal data to determine the type of the predicted seizure comprises:
identifying one or more seizure onset signal patterns in the received neuronal electrical activity signal data;
generating a predicted seizure metric from the one or more seizure onset signal patterns, wherein the predicted seizure metric is a probability metric indicating a probability of the presence of each of the plurality of predicted seizure types; and
determining the type of the predicted seizure based on the predicted seizure metric.

4. The method of claim 1, wherein:
the analyzing the received neuronal electrical activity signal data to determine the type of the predicted seizure comprises:
identifying one or more seizure onset signal patterns in the received neuronal electrical activity signal data, wherein the one or more seizure onset signal patterns are pre-bifurcation signal patterns.

5. The method of claim 1, wherein:
the analyzing the received neuronal electrical activity signal data to determine the type of the predicted seizure comprises:
identifying one or more seizure onset signal patterns in the received neuronal electrical activity signal data, wherein the one or more seizure onset signal patterns include one or more signal bifurcation patterns.

6. The method of claim 1, wherein the analyzing the received neuronal electrical activity signal data to determine the type of the predicted seizure comprises:
determining a plurality of seizure metrics, each seizure metric of the plurality of seizure metrics corresponding to a particular type of predicted seizure; and
determining the type of predicted seizure according to the determined plurality of seizure metrics.

7. The method of claim 1, wherein:
the analyzing the received neuronal electrical activity signal data to determine the type of the predicted seizure comprises: (i) determining a predicted seizure metric, and (ii) determining the type of the predicted seizure based on the predicted seizure metric;
the method further comprises:
in response to administering the treatment electrical stimulation pattern to the subject through the plurality of electrodes, receiving, in the signal processing device, further neuronal electrical activity signal data taken from a plurality of electrodes;
determining, in the signal processing device, an increase, decrease, or no change in the predicted seizure metric based on the further neuronal electrical activity signal data and generating an updated predicted seizure metric; and
in response to the determination of the increase, decrease, or no change in the predicted seizure metric, changing the treatment electrical stimulation to reduce the likelihood of the predicted seizure.

8. The method of claim 1, further comprising:
prior to the sending of the instructions to administer the probing stimulation pattern, receiving, via the signal processing device, training neuronal electrical activity signal data taken from the plurality of electrodes; and
training, via the signal processing device, a model of the subject based on the received training neuronal electrical activity signal data;
wherein the determination of the type of predicted seizure is further based on the trained subject model of the subject by: (i) inputting the neuronal electrical activity signal data into the trained patient model, and (ii) receiving the type of predicted seizure as an output of the trained patient model.

9. The method of claim 1, further comprising administering the probing electrical stimulation pattern and/or the treatment electrical stimulation pattern to the subject through the plurality of electrodes.

10. The method of claim 1, wherein the type of the predicted seizure is one of:
a supercritical Hopf bifurcation (SupH) bifurcation;
a Saddle-Node on an Invariant Circle (SNIC) bifurcation;
a Saddle-Node (SN) bifurcation; or
a Subcritical Hopf (SubH) bifurcation.

11. The method of claim 1, wherein the probing electrical stimulation pattern comprises at least one of:
a brain location to stimulate;
amplitude of the probing electrical stimulation pattern;
frequency of the probing electrical stimulation pattern;
duration of the probing electrical stimulation pattern; or
start time of the probing electrical stimulation pattern.

12. The method of claim 1, wherein the treatment electrical stimulation pattern comprises at least one of:
a brain location to stimulate;
amplitude of the treatment electrical stimulation pattern;
frequency of the treatment electrical stimulation pattern;
duration of the treatment electrical stimulation pattern; or
start time of the treatment electrical stimulation pattern.

13. The method of claim 1, wherein the plurality of electrodes include at least one electrode configured to both: (i) administer at least part of the probing electrical stimulation pattern, and (ii) sense neuronal electrical activity.

14. The method of claim 1, wherein the plurality of electrodes includes: (i) an administration electrode configured to administer at least part of the probing electrical stimulation pattern, but not configured to sense neuronal electrical activity; and (ii) a sensing electrode configured to sense the neuronal electrical activity, but not configured to administer the probing electrical stimulation pattern.

15. The method of claim 1, wherein the plurality of electrodes are one or both of: (i) comprised in a therapeutic brain implant, or (ii) extracranial.

16. The method of claim 1, wherein the sending of the instructions to administer the probing electrical stimulation pattern occurs subsequent to a first seizure.

17. A method comprising:
sending, by a signal processing device, instructions to administer a probing electrical stimulation pattern to a subject through a plurality of electrodes;
receiving, at the signal processing device, neuronal electrical activity signal data taken from the plurality of electrodes in response to the sent instructions to administer the probing electrical stimulation pattern;
analyzing, in the signal processing device, the received neuronal electrical activity signal data and, from the received neuronal electrical activity signal, determining a type of predicted seizure from a plurality of seizure types; and
determining, in the signal processing device, from the type of predicted seizure whether to administer a treatment electrical stimulation pattern to prevent the predicted seizure.

18. The method of claim 17, wherein:
the analyzing the received neuronal electrical activity signal data to determine the type of the predicted seizure comprises:
identifying one or more seizure onset signal patterns in the received neuronal electrical activity signal data;
generating a predicted seizure metric from the one or more seizure onset signal patterns, wherein the predicted seizure metric spans a plurality of value ranges each value range corresponding to a different one of the plurality of predicted seizure types; and
determining the type of the predicted seizure based on the value range of the predicted seizure metric.

19. The method of claim 17, wherein:
the analyzing the received neuronal electrical activity signal data to determine the type of the predicted seizure comprises:
identifying one or more seizure onset signal patterns in the received neuronal electrical activity signal data;
generating a predicted seizure metric from the one or more seizure onset signal patterns, wherein the predicted seizure metric is a probability metric indicating a probability of the presence of each of the plurality of predicted seizure types; and
determining the type of the predicted seizure based on the predicted seizure metric.

20. The method of claim 17, wherein:
the determining whether to administer the treatment electrical stimulation pattern comprises determining to administer the treatment electrical stimulation pattern; and
the method further comprises determining the treatment electrical stimulation pattern based on the determined type of predicted seizure, wherein the treatment electrical stimulation pattern comprises at least one of:
a brain location to stimulate;
amplitude of the treatment electrical stimulation pattern;
frequency of the treatment electrical stimulation pattern;
duration of the treatment electrical stimulation pattern; or
start time of the treatment electrical stimulation pattern.

21. The method of claim 17, wherein the determining whether to administer the treatment electrical stimulation pattern comprises determining not to administer the treatment electrical stimulation pattern to prevent the predicted seizure, and wherein the method further comprises:
in response to the determination that the treatment electrical stimulation pattern will not be administered, sending a warning message to a smartphone of a subject indicating: (i) the type of the predicted seizure, and (ii) that no electrical stimulation will be administered to prevent an onset of the predicted seizure.

22. A method comprising:
receiving, via one or more processors, training neuronal electrical activity signal data taken from a plurality of electrodes;
receiving, via the one or more processors, medication data of a subject, wherein the medication data includes a type of a medication, a dosage amount of the medication, and a time that the medication was administered to the subject; and
training, via the one or more processors, a subject model of the subject based on: (i) the received training neuronal electrical activity signal data, and (ii) the medication data of the subject;
wherein the subject model is operable to determine a type of seizure based on subsequent neuronal electrical activity signal data.

23. The method of claim 22, further comprising:
receiving, via the one or more processors, brain state data of the subject.

24. The method of claim 23, wherein the brain state data includes at least one of:
sleep stage data of the subject or circadian rhythm data of the subject.

25. The method of claim 22, wherein the type of the predicted seizure is one of:
a supercritical Hopf bifurcation (SupH) bifurcation;
a Saddle-Node on an Invariant Circle (SNIC) bifurcation;
a Saddle-Node (SN) bifurcation; or
a Subcritical Hopf (SubH) bifurcation.

26. The method of claim 22, wherein the one or more processors are comprised in a signal processing device, and the method further includes:
sending, by the signal processing device, instructions to administer a probing electrical stimulation pattern to the subject through the plurality of electrodes;
in response to the sending of the instructions to administer the probing electrical stimulation pattern, receiving, at the signal processing device, the subsequent neuronal electrical activity signal data;
determining, in the signal processing device, a type of the predicted seizure by inputting the subsequent neuronal electrical activity signal data into the trained model;
determining, in the signal processing device, a treatment electrical stimulation pattern to prevent the predicted seizure based on the type of the predicted seizure;
administering, through the plurality of electrodes, the treatment electrical stimulation pattern; and
updating, in the one or more processors, the subject model based on neuronal electrical activity signal data taken from the plurality of electrodes in response to the administered treatment electrical stimulation pattern.
